(19) United States
(12) Patent Application Publication
(10) Pub. No.: US 2024/0058580 A1
(43) Pub. Date: Feb. 22, 2024

(54) MAGNETIC RESONANCE IMAGING GUIDED ACTIVE INJECTION NEEDLE FOR RADIATION-ONCOLOGY BRACHYTHERAPY

(71) Applicant: THE JOHNS HOPKINS UNIVERSITY, Baltimore, MD (US)

(72) Inventors: Akila VISWANATHAN, Baltimore, MD (US); Yue CHEN, Fayetteville, AR (US); Anthony GUNDERMAN, Fayetteville, AR (US); Henry R. HALPERIN, Baltimore, MD (US); Ehud J. SCHMIDT, Towson, MD (US); Ryan BAUMGAERTNER, Baltimore, MD (US); Marc MORCOS, Baltimore, MD (US)

(73) Assignee: THE JOHNS HOPKINS UNIVERSITY, Baltimore, MD (US)

(21) Appl. No.: 18/257,189

(22) PCT Filed: Dec. 13, 2021
(86) PCT No.: PCT/US2021/063037

§ 371 Date: Jun. 13, 2023

Related U.S. Application Data
(60) Provisional application No. 63/126,319, filed on Dec. 16, 2020.

(51) Int. Cl. A61M 25/01; A61M 25/06; A61M 5/32; A61N 5/10

(52) U.S. Cl. CPC A61M 25/0105; A61M 25/06; A61M 5/329; A61N 5/1007; A61N 5/1027; A61N 5/1049; A61N 2005/1055

(57) ABSTRACT

A substantially metallic magnetic resonance Imaging (MRI)-tracked injection needle device is disclosed. The magnetic resonance Imaging (MRI)-tracked injection needle device includes a luer syringe; an electrical connector that is at least partially housed in an interior space of a distal end of the luer syringe; an electrical adaptor coupled to the electrical connector; and an injection needle comprising a shaft having a needle distal end and a needle proximal end, the shaft comprising concentric metal tubes comprising an inner metal tube and an outer metal tube, the needle proximal end coupled to the electrical adaptor and the needle distal end comprising one or more tracking coils arranged between the inner metal tube and the outer metal tube.

### CROSS-REFERENCE TO RELATED APPLICATIONS

This application claim priority to U.S. Provisional Patent Application Ser. No. 63/126,319 filed on Dec. 16, 2020, the disclosure of which is incorporated herein by reference in its entirety.

### GOVERNMENT RIGHTS

This disclosure was made with Government support under Contract No. RO1 CA237005-01A1 awarded by the National Institutes of Health. The Government has certain rights in the invention.

### FIELD

The present teachings generally relate to magnetic resonance imaging (MRI)-guided active injection needle for radiation-oncology brachytherapy.

### BACKGROUND

The goal of effective radiation treatment of tumors is to provide maximal radiation dose to all parts of the tumor, while sparing radiation from surrounding normal tissues. In some cases, there are surrounding tissues which are especially sensitive to radiation, or the tissues are very close to the treated region so that it is difficult to reduce the dose they receive. To resolve the above issues, it is now possible to insert materials in order to increase the space between the tumor and the tissues that should be spared. A common way to do that is to inject polymer hydrogels or liquids into specific interfaces between tissues, so as to create anatomic “pockets” that are filled with this material, which then serves to distance the sensitive tissue and reduce the received radiation dose. These biocompatible polymer hydrogels are intended to be absorbed by the body once the radiation delivery procedure is complete. There are several hydrogels which are currently regulatory approved in Europe and the US. For further details see “Hydrogel Spacer Application Technique, Patient Tolerance and Impact on Prostate Intensity Modulated Radiation Therapy: Results from a Prospective, Multicenter, Pivotal Randomized Controlled Trial” by Pieczonka C M, Urology Practice 2016, Vol 3 141-146 or “Application technique: placement of a prostate—rectum spacer in men undergoing prostate radiation therapy” by Hatiboglu G, Brit. Jour. Urology International (BJUI) 2012 E647-E652.

The goal of successful pre-procedural injection is to insert the hydrogel into the correct location and establish that the dimensions of the pocket meet the radiation dose reduction by spacing requirements. This task is commonly accomplished by using an imaging modality that (a) detects the position of the injection needle, (b) relative to its desired final location, and (c) can show the topology of the injected pocket. The desire is that this needle deployment procedure; (I) require a minimal amount of time, (II) provide optimal visualization of the needle position during its navigation to the target region, and (III) show the hydrogel pocket's topology (volume, shape) during the filling of the pocket. Hydrogels are used in brachytherapy, in which spatially-localized radiation sources are inserted into the tumor and its surroundings, in order to further distance sensitive non-cancerous normal tissues from the radiation dose. Hydrogels are also used in external beam radiation therapy (EBRT) with the same rationale, to distance non-cancerous normal tissue structures, called organs at risk (OARs).

Similar situations exist for thermal ablations performed by Interventional Radiologists. In these cases, special invasive probes are used to deliver thermal or cooling (cryogenic) energy to specific tumor regions, but this energy tends to diffuse or flow away from the delivery location, and can therefore damage surrounding tissues. Here again, it is possible to place pockets of hydrogel that have a low thermal conduction, so that the propagation of the thermal (or cooling) front be steered away from sensitive tissues. Additional analogous situations occur during the injection of chemotherapy agents into tumors through their feeding blood vessels, and/or to the blocking of specific blood vessels with embolizing particles.

In most situations, the interventional devices employed have a very small (<2 mm) diameter, in order to minimize tissue puncture injury, and typically have a length/diameter ratio>>1, which is required in order to manipulate them from outside the body (i.e. from their proximal end). The best materials from which these devices are constructed are metals with large (Young's and Torsional) elastic moduli such as stainless steel or titanium.

Most of these injection procedures are today performed under X-ray or Ultrasound (ULS) guidance. Magnetic Resonance Imaging (Mill) sequences can be used to visualize the above procedures, but this is not commonly performed. This is because (A) Mill use requires use of MRI-conditional devices, which are devices that can be safely used inside the MRI scanner, which restricts the material choices (ferromagnetic and high paramagnetic materials cannot be used), restricts the geometry of the devices (lengths larger than an Mill Radio-frequency quarter-wavelength typically are not used), and may require the use of materials with suboptimal mechanical properties. (B) Passive spatial localization (“tracking”) of devices, which entails seeing the needles without adding special sensors onto the device shaft, is easy to perform with X-ray or ULS monitoring, but it is quite time inefficient when used with Mill, since it is difficult to accurately locate metallic devices in the Mill scanner without employing high resolution sequences (which require long imaging times). The image processing to obtain the needle location also requires a significant amount of time, which adversely affects the overall clinical workflow.

In one prior invention, we developed an updated version of the MR-Tracking pulse sequence that can be used in conjunction with metallic devices. That invention encompassed the use of (a) flexible printed-circuit (FPC) MRI micro-coils with a lobe pattern which is principally oriented perpendicular to the shaft direction, and therefore are well seen on the metallic devices, and (b) addition of phase-dithering gradient lobes to the sequence which reduce localization artifacts that result from the metallic surfaces. We are therefore able to perform precise (˜0.6×0.6×0.6 mm³) and fast (15 frames per second) localization of the MR-tracking micro-coils. Real-time active MR-tracking of metallic stylets in MR guided radiation therapy by Wang W et al. Magn. Reson. Med. 2015 May; 73(5): 1803-1811. Patent is “An Active Tracking System and Method for MRI” US-2015-0338477-A1, assigned to BWH.

In a second prior invention, we developed a special device, the MBalun, which when overlaid on the metal surface, reduce the currents that are induced on the metallic devices by the MRI scanner's body coil. After overlaying one or more MBaluns on the shaft, we are now able to build very long (2 m) metallic devices that do not heat beyond FDA/IEC limits during the highest Specific Absorption Rate (SAR) sequences, and are therefore MRI-safe. Patent Title: “MRI Radio-Frequency Heating Amelioration For Metallic braided Catheters” WO 2019/040395 A1 Published 28.02.2019. Assigned to JHU. Paper is “MRI Conditional Actively-Tracked Metallic Electrophysiology Catheters and Guidewires with Miniature Tethered Radio-Frequency Traps: Theory, Design and Validation by Alipour A et al. IEEE Trans Biomed Eng. 2020 June; 67(6):1616-1627.

### SUMMARY

In accordance with examples of the present disclosure, a magnetic resonance Imaging (MRI)-tracked injection needle device is disclosed. The magnetic resonance Imaging (MRI)-tracked injection needle device comprises a Luer lock connector for syringes; an electrical connector that is at least partially housed in an interior space of a distal end of the Luer lock connector for syringes; an electrical adaptor coupled to the electrical connector; and an injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip and comprising one or more tracking coils arranged between the inner metal tube and the outer metal tube.

Various additional features of the magnetic resonance Imaging (MRI)-tracked injection needle device can be included such as one or more of the following. The needle distal end further comprising two holes cut into the outer metal tube to permit MRI signals to be obtained from more than one orientation of the injection needle. The tip is a beveled tip. The beveled tip has a 30° bevel. The needle distal end further comprises flexible printed circuit board connected by cables to one or more tracking coils. The one or more tracking coils provide electrical tracking signals representative of location information of the tip and the flexible printed circuit board transmits the electrical tracking to a receiver of a MRI scanner. The needle distal end further comprising a protective heat shrink to provide water insulation for the one or more tracking coils and cables. The concentric metal tubes are composed of titanium. The inner metal tube has dimensions of 0.81 mm×0.10 mm and the outer metal tube has dimensions of 1.62 mm×0.2 mm. The magnetic resonance Imaging (MRI)-tracked injection needle device further comprises an insulator layer arranged on an exterior surface of the outer metal tube. The shaft further comprises a solenoid that is connected in series to a thin film capacitor that is separated by the insulator layer with one end of the solenoid and one end of the thin film capacitor to the outer metal tube to reduce MRI radio-frequency induced heating of the shaft.

In accordance with examples of the present disclosure, a method for fabricating a magnetic resonance Imaging (MRI)-tracked injection needle device is disclosed. The method comprises attaching an injection needle to a syringe, the injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip; disposing at least one coil of electrical conductor between the inner metal tube and the outer metal tube; and providing an electrical output to at least one coil.

In accordance with examples of the present disclosure, a method for using a tracking system for magnetic resonance imaging (MRI) is disclosed. The method comprises electrically connecting an injection needle to electronic circuitry, the injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip and comprising a first tracking coil and a second tracking coil arranged between the inner metal tube and the outer metal tube; generating sequences of MRI-pulses to acquire, with an MRI system, projection data representing three one-dimensional projections of the injection needle along three orthogonal spatial axes such as to determine a three-dimensional position of the at least one coil of the first tracking coil and the second tracking coil from sequenced projection data; and generating data representing a position of the tip by extrapolating the projection data along a direction connecting positions of the first tracking coil and the second tracking coil.

In accordance with examples of the present disclosure, a method for reducing the deviation of the needle trajectory from the clinician's planned linear trajectory in the body is disclosed. In practical cases, there are several tissues that the needle needs to traverse between the entry point in the skin and the target, which can be several cm inside the tissue. Depending on several factors, such as the angle of insertion into a certain tissue relative to the respective tissue's principle symmetry directions, and/or if the elastic moduli of a tissue is large enough relative to the elastic moduli of the needle, and/or if the elastic moduli of the tissues is anisotropic, and also depending on the relative dimensions of the needle (such as its length and radius, and the sharpness of its tip), the forces applied by the surrounding tissues can be unbalanced on opposing surfaces of the needle, leading to the deflection of the needle path from its planned trajectory. In the present disclosure, the deflection angles of the trajectory are reduced by cutting specifically-defined groves into the needle's outer surface (e.g. serrating it's surface). Proper determination of these incremental groves such as the distance between successive groves, their depth and width, and the sharpness of the angles in the grove, leads to a situation where pushing down on the needle leads to a division of the force applied by the surrounding tissues by the number of serrations. As a result, different numbers of serrations will incur varying amounts of frictional force during insertion of the needle. In addition, partial reduction of the contact area with the surrounding tissues occurs in the serrated regions, which leads to a redistribution of the forces exerted by the needle along its shaft (e. g. higher pressure at the contact points), which in turn, varies the work done to advance the needle. Finite Element Analysis (FEA) simulations can therefore be used to reduce the trajectory deviation.

On the other hand, removing (cutting-out) materials from the needle surface in a different fashion can also lead to a desired curved trajectory, which can be helpful in creating a nonlinear path to avoid critical structures such as blood vessels, or nerves, while arriving at a desired target. The FEA simulation is able to predict the needle deformation during the insertion process for a given cut-out pattern. Thus, we can leverage the FEA calculation's result to optimize the needle design to create either linear trajectories or nonlinear trajectories, depending on the requirement.

### BRIEF DESCRIPTION OF THE DRAWINGS

The accompanying drawings, which are incorporated in, and constitute a part of this specification, illustrate implementations of the present teachings and, together with the description, serve to explain the principles of the disclosure. In the figures:

FIG. 1 shows a layout of the MR-tracked injection needle according to examples of the present disclosure.

FIG. 2 shows longer injection needles that are mounted MBaluns at incremental distances along their shaft according to examples of the present disclosure.

FIG. 3 shows an expanded view of the distal MR-tracked injection needle according to examples of the present disclosure.

FIG. 4A shows a picture of the tip of the MR-tracked injection needle of FIG. 1.

FIG. 4B shows another picture of the tip of the MR-tracked injection needle of FIG. 1.

FIG. 4C shows a picture of the handle of the MR-tracked injection needle of FIG. 1.

FIG. 5 shows an electrical schematic of the tuning matching circuit in the handle of the MR-tracked injection needle of FIG. 1 according to examples of the present disclosure.

FIGS. 6A, 6B, 6C, and 6D show a series of images for the visualization of injection needle navigation in a cervical cancer phantom on the 3D Slicer user interface, taken at one time point during the navigation according to examples of the present disclosure.

FIG. 7 is a series of images showing water coming out of the beveled tip and spreading forward and to the left over time that was performed in a cervical cancer phantom.

FIG. 8A-8D show a series of pictures of the MR-Tracked injection needle according to examples of the present disclosure, where FIG. 8A shows a complete 24 cm long and 1.6 mm outer diameter needle, FIG. 8B shows an expanded distal end showing the MRT coils and beveled tip, FIG. 8C shows an expanded handle, showing syringe and quick-disconnect electrical connectors, and FIG. 8D shows a GRE MRI image of needle tip, showing hyperintense beveled tip and MRT coils. The tip hyperintensity and the dark susceptibility artifacts proximal to the MRT coils are caused by the cut metal surfaces, are seen only with GRE and can be reduced by coating the metal with a paramagnetic metallic film (e.g. “susceptibility matching”).

FIGS. 9A and 9B show a series of pictures of MR-guided navigation in a swine, where FIG. 9A shows T2-weighted images acquired before navigation and injection show the region of the vaginal cavity, with an inserted vaginal obturator, and rectum, and FIG. 9B shows Images obtained after injection of GDTPA-doped water between the vaginal cavity and the rectum show the topology of the created pocket.

FIG. 10A shows a side view of the outer tube during the machining operations that are shown in FIG. 10B-FIG. 10D according to examples of the present disclosure.

FIG. 10B shows an epoxy adhesive placement operation according to examples of the present disclosure.

FIG. 10C shows a machining of the beveled tip operation according to examples of the present disclosure.

FIG. 10D shows the ends with the inclusion of the MRT circuitry, including a distal coil, a proximal coil, and coaxial cables according to examples of the present disclosure.

FIG. 10E shows a cross-section of the MR-tracked needle at the distal end, showing coaxial cables, outer tube, inner tube, tracking coils, and epoxy according to examples of the present disclosure.

FIG. 10F shows a side view of the proximal end of the needle assembly according to examples of the present disclosure.

FIG. 11 shows an electrical adapter with the injection needle assembly of FIG. 10E and FIG. 10F.

FIG. 12A shows the FPC tracking coils attached to the distal end of the needle according to examples of the present disclosure.

FIG. 12B shows the tuning and matching circuit schematic according to examples of the present disclosure.

FIG. 12C shows the results of the tuning and matching of the circuit for each coil, indicating strong signal transmission according to examples of the present disclosure.

FIG. 12D shows a side view of injection needle and electrical adapter according to examples of the present disclosure.

FIG. 13 shows a simulation result of the 30 cm long needle entering tissue vertically downwards from the top (with 12.7 mm long grooves) when the insertion depth is set to 30 mm according to examples of the present disclosure.

FIG. 14A shows the simulated needle tip deviation from the linear insertion path is plotted with respect to the insertion depth according to examples of the present disclosure.

FIG. 14B shows the visual simulation results of a needle shown in FIG. 14A without grooves (FIG. 14A) and FIG. 14C shows the visual simulation results of a needle shown in FIG. 14A with grooves depicted within the tissue with respect to the von Mises stress according to examples of the present disclosure.

It should be noted that some details of the figures have been simplified and are drawn to facilitate understanding of the present teachings rather than to maintain strict structural accuracy, detail, and scale.

### DETAILED DESCRIPTION

Generally speaking, examples of the present disclosure provide for an MRI-tracked metallic injection needle. This needle is intended to be used for injecting a hydrogel into tissue pockets, so as to prevent sensitive normal tissues from receiving the radiation dose that is delivered to the neighboring tumor. The immediate design targets are needles for transperineal polymer hydrogel injections, which are used during the course of external beam or brachytherapy treatments of gynecologic and prostate cancer, as well as other cancers. The needle is intended to be used for injecting a hydrogel into regions between the tumor and normal tissues, so as to prevent sensitive normal tissues from receiving the radiation dose that is delivered to the neighboring tumor. The immediate design targets are needles for transperineal polymer hydrogel injections, which are used during the course of brachytherapy treatments of gynecologic and either external beam or brachytherapy for prostate cancer or other cancers such as pancreatic cancer.

The needle may be; (I) MRI-conditional, (II) possess similar mechanical properties to conventional (non-MRI-conditional) stainless-steel needles, (III) allow rapid navigation from the skin insertion point to the target area using MR-tracking of miniature tracking coils placed close to the needle tip.

Note that similarly designed needles can be used in order to inject a variety of agents into the vascular or soft-tissue space.

A variant on this design can be used for tissue suction into the inner tube (e.g. by applying a negative pressure) which can serve for biopsy purposes.

Similar devices may be used for: (1) MRI-guided injections of (Low thermal conduction) blocking liquids or hydrogel that prevent the spread of heat or cold fronts from thermal (Radio-frequency, Microwave, focused ultrasound) sources or cooling (cryoablation) sources to unwanted anatomy; (2) MRI-guided deployment of chemotherapy agents; and (3) MRI guided deployment of vessel blockage (or embolization) particles or adhesives.

Additional advantages of the MR-guide injection needles include (1) rapid tracking of the needle, so that it can be brought and placed in the correct location quickly. In some instances, this can also reduce the need to make multiple holes in the soft tissue and (2) localization of the needle tip allows easy subsequent monitoring of the process of hydrogel/liquid injection by performing sequential imaging in a region around the final tip location.

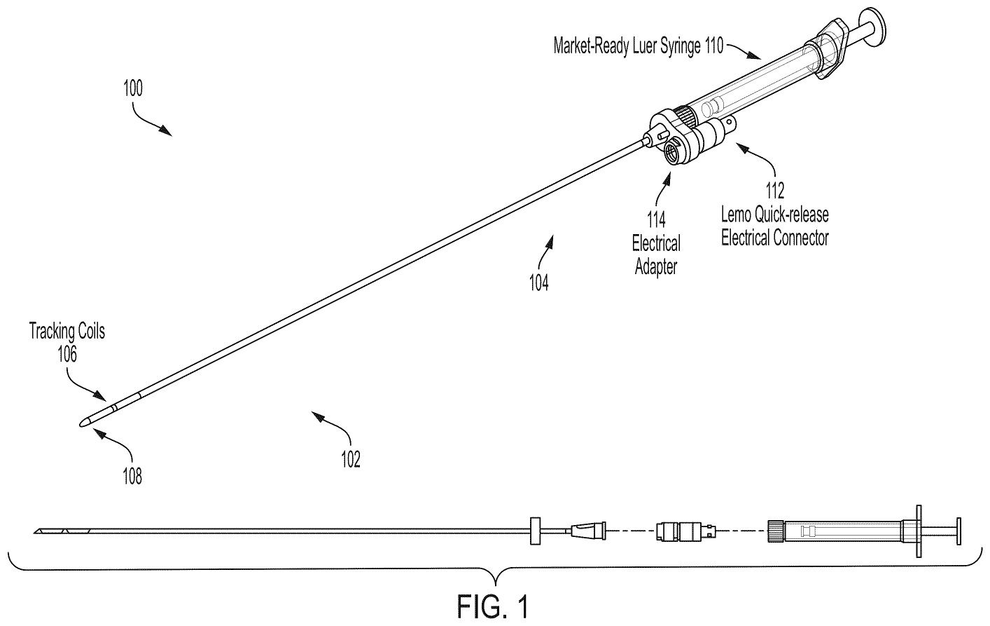

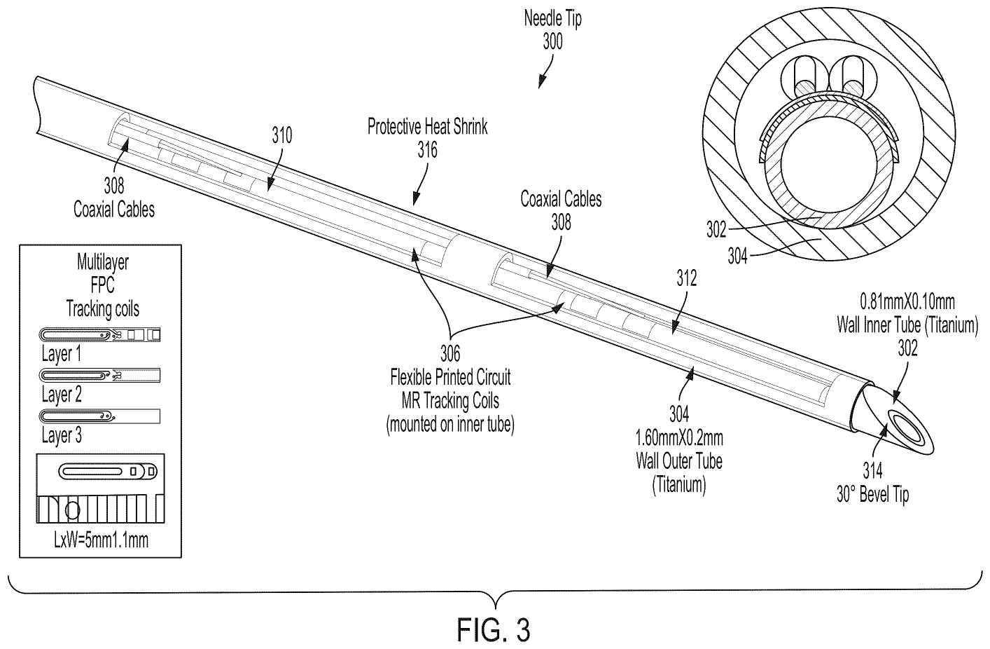

FIGS. 1-3 provide the technical details of the MR-tracked injection needles. FIG. 1 shows a layout of the MR-tracked injection needle 100 according to examples of the present disclosure. By one non-limiting example, MR-tracked injection needle 100 can be made of concentric titanium tubes. Other suitable materials can be used, such as Austenitic Stainless Steel, Gold, Inconel R (Inco Alloys, Inc., a Nickel-Chromium alloy), Nitinol, Beryllium Copper, Tungsten, Bronze, Aluminum Bronze. MR-tracked injection needle 100 comprises distal end 102 and proximal end 104. At distal end 102, MR-tracked injection needle 100 comprises tracking coils 106, such as two MR-tracking coils, which detect the location and orientation of tip 108. At proximal end 104, MR-tracked injection needle 100 is connected to syringe 110, such as an ordinary syringe, from which the hydrogel and connected to electrical connector 112 and electrical adaptor 114, which are used to transfer the MR Tracking signals onwards to the receivers of the MRI scanners, passing first through tuning, matching and decoupling circuits and then going through current isolation circuits. To allow tracking coils 106, at distal end 102 to get MRI signals from the MR spins, which are outside the device, two rectangular holes (“windows) can be cut in the outer titanium tube, as shown in FIG. 3. Each of the micro-coaxial cables runs through its own lumen, primarily in order to prevent strain on the cables. For example, for cervical cancer and prostate cancer, 9.5 and 24 cm needle lengths are used, respectively.

The two concentric tubes of MR-tracked injection needle 100 comprises an inner tube that is used as the (hydrogel/liquid) injection lumen and an outer tube that holds the entire device together, and provides additional torsional and compressive (Young's modulus) mechanical strength to the needle, which is useful for allowing it to be advanced through tissue without bending. The two tubes can be set in place using epoxy. The entire needle, excluding the distal tip, can be coated on its exterior by a thin heat-shrink tube, which makes the structure water proof, thus preventing damage to tracking coils 106 and the cables inside. The area between the inner tube and the outer tube is used for the tracking coils 106, e.g., two MR-tracking coils, and their signal-transmitting microcoaxial cables. The tracking coils are mounted on the internal tube, with an approximately 100 micron layer of insulating epoxy separating the metallic tube from the coils. The metallic regions, including the tip, can optionally be covered with diamagnetic (copper, silver, gold) metallic films to reduce magnetic susceptibility artifacts that may distort MRI images in areas directly adjacent to the metallic shaft and tip.

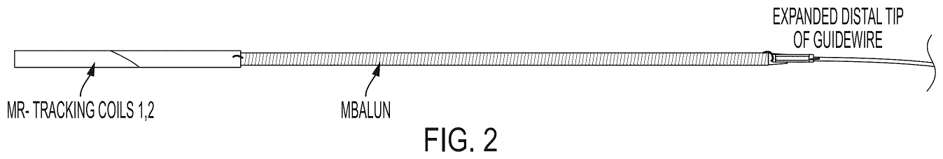

If very long injection (greater than half an electromagnetic wavelength in the surrounding fluid) needles are required, MBaluns can be added at selected increments around the outer titanium tube and soldered to the outer titanium tube, or placed into dedicated groves cut into the external titanium tube. These MBaluns typically enlarge the outer diameter by ˜0.1 mm. FIG. 2 shows a longer injection needle that is mounted with MBaluns at incremental distances along their shaft according to examples of the present disclosure. The MBaluns on the longer injection needle can at least reduce, if not prevent, MRI radio-frequency induced heating of the shaft, such as a titanium shaft, and bright it to within regulatory limits. Each MBalun is constructed from a loosely wound solenoid that is connected in series to a thin-film capacitor. The MBalun is separated from the outer titanium tube by a thin insulator, with one end of the solenoid and one end of the capacitor soldered to the titanium tube.

FIG. 3 shows an expanded view of the distal MR-tracked injection needle 300 according to examples of the present disclosure. MR-tracked injection needle 300 can be the needle at distal end 102 and is built of two concentric (0.8 and 1.6 mm Outer Diameter) titanium tubes, comprising inner tube 302 and outer tube 304. Inner tube 302 is used as the injection lumen and is mounted at its distal end with the two Flexible Printed Circuit (FPC) MR-tracking coils 306, and further proximally with the two micro-coaxial cables 308 that transmit the MRT signal to the proximal end of the needle. Outer tube 304 comprises two distal rectangular windows 310, 312 above the locations of MR-tracking coils 306, which allow MR-tracking coils 306 to detect the MR signals from the surrounding liquid, and thus provide location information about the location and orientation of needle tip 314. The entire needle, except for a 10 mm area at the distal tip, is coated with an insulator (heat shrunk) film 316 to provide water insulation for MR-tracking coils 306 and micro-coaxial cables 308. The 250 μm thick FPC MR-tracking coils 306 are tuned and matched internally using embedded (single-layer thin film) capacitors.

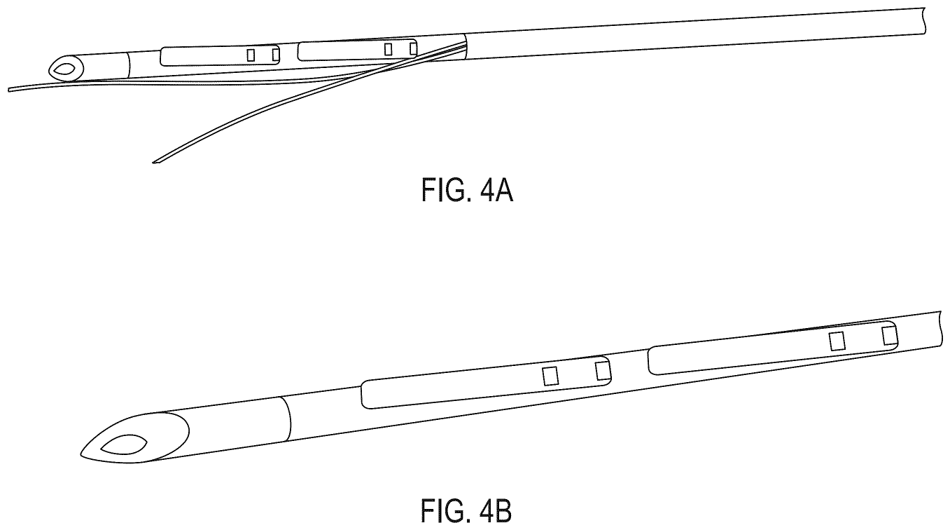

FIG. 4A shows a picture of the tip of the MR-tracked injection needle of FIG. 1. FIG. 4B shows another picture of the tip of the MR-tracked injection needle of FIG. 1. FIG. 4C shows a picture of the handle of the MR-tracked injection needle of FIG. 1.

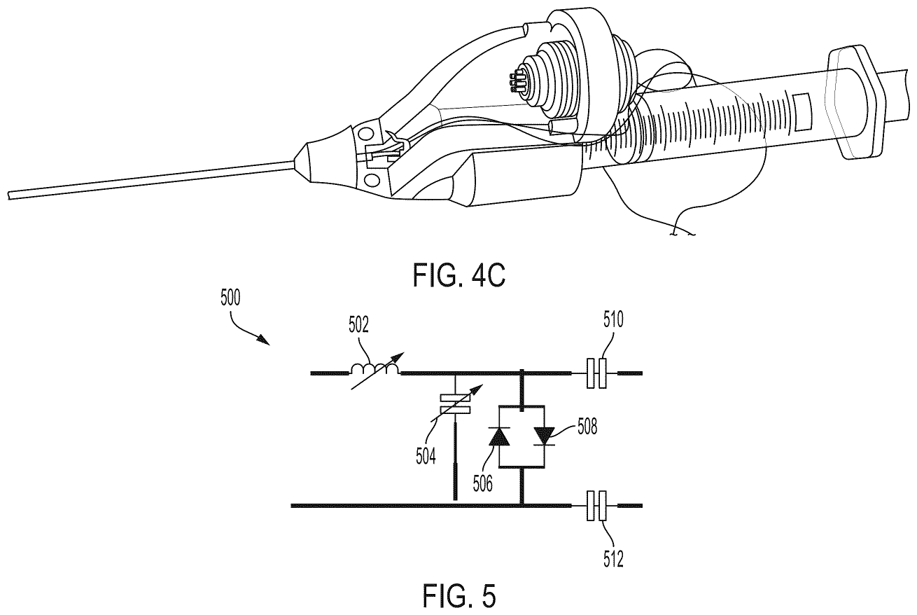

FIG. 5 shows an electrical schematic of the tuning matching circuit 500 in the handle of the MR-tracked injection needle of FIG. 1 according to examples of the present disclosure. A tuning matching circuit 500 is connected to both MR tracking coils 106, 306 and are tuned, matched, and decoupled from each other with a series variable inductor 502, for example a 65-99 pf variable inductor, and a parallel variable capacitor 504, for example a 68 pF parallel variable capacitor, anti-parallel decoupling diodes 506 and 508, and blocking capacitors 510 and 512, for example 900 pF 0.5 KV blocking capacitors.

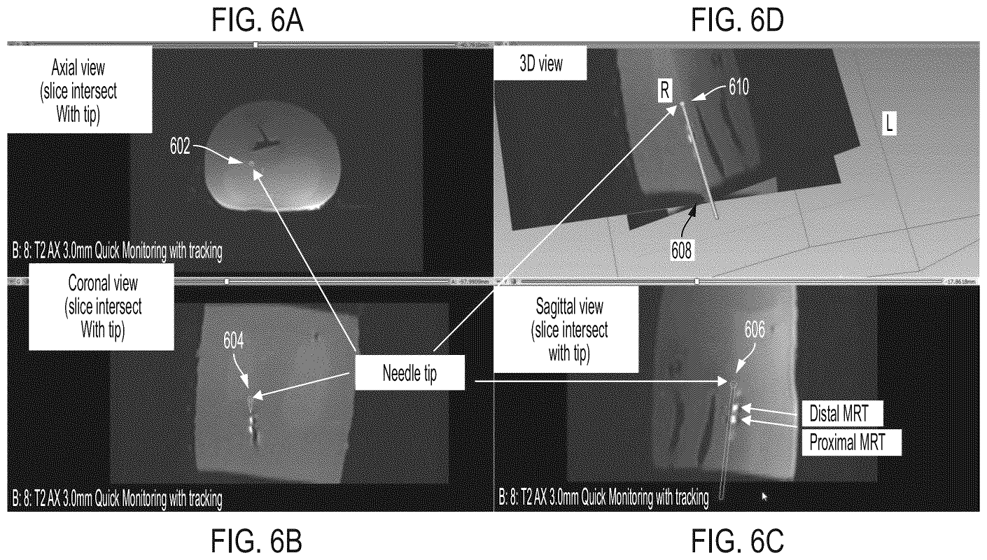

FIGS. 6A, 6B, 6C, and 6D show a series of images for the visualization of injection needle navigation in a gynecologic cancer phantom on the 3D Slicer user interface, taken at one time point during the navigation according to examples of the present disclosure. The background images are taken from an MRI T1-weighted data set covering the entire phantom, which was acquired using the scanner's surface coils together with the MRT coils. FIG. 6A is an axial view, FIG. 6B is a coronal view, FIG. 6C the bottom right is a sagittal view, and FIG. 6D is a 3D view (a display of the axial, sagittal and coronal slices, showing the location where the 3 slices intersect, which can be rotated to show the information from various directions). FIG. 6A shows an axial slice from within the MRI dataset that is located in the same plane as the instantaneous axial location of the needle tip, as provided by the MRT Tracking information. The dot 602 indicates the actual location, within the slice, of the tip. FIG. 6B shows a coronal slice from within the MRI dataset that is located in the same plane as the instantaneous coronal location of the needle tip, as provided by the MRT Tracking information. The dot 604 indicates the actual location, within the slice, of the tip, while the empty lines show the orientation of the needle trajectory proximal to the tip, as provided by the MRT Tracking information. FIG. 6C shows a sagittal slice from within the MRI dataset that is located in the same plane as the instantaneous coronal location of the needle tip. The dot 606 indicates the actual location, within the slice, of the tip, while the empty lines show the orientation of the needle trajectory proximal to the tip. It is also possible to see in this picture the actual hyperintense signals of the two (distal, proximal) MRT coils themselves. In FIG. 6D, it is possible to see in the complete instantaneous trajectory of the needle 608, with the circle 610 indicating its tip location at this time.

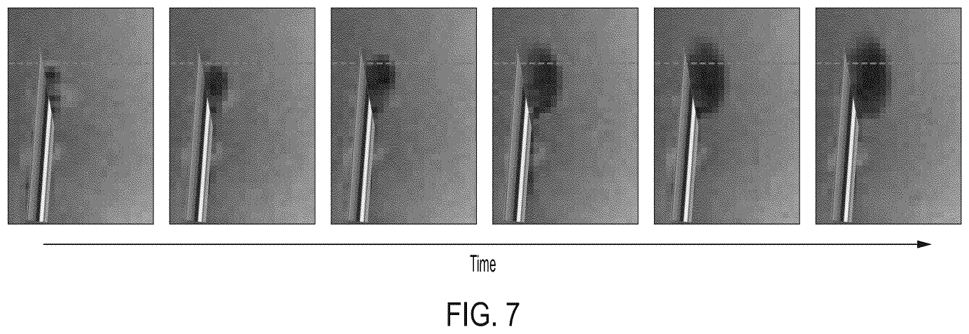

FIG. 7 is a series of MRI images taken at increasing times that show water coming out of the beveled tip and spreading forward and to the left. These were acquired during an experiment that was conducted in a gynecologic cancer phantom, illustrating use of the injection needle for injecting gel or fluid.

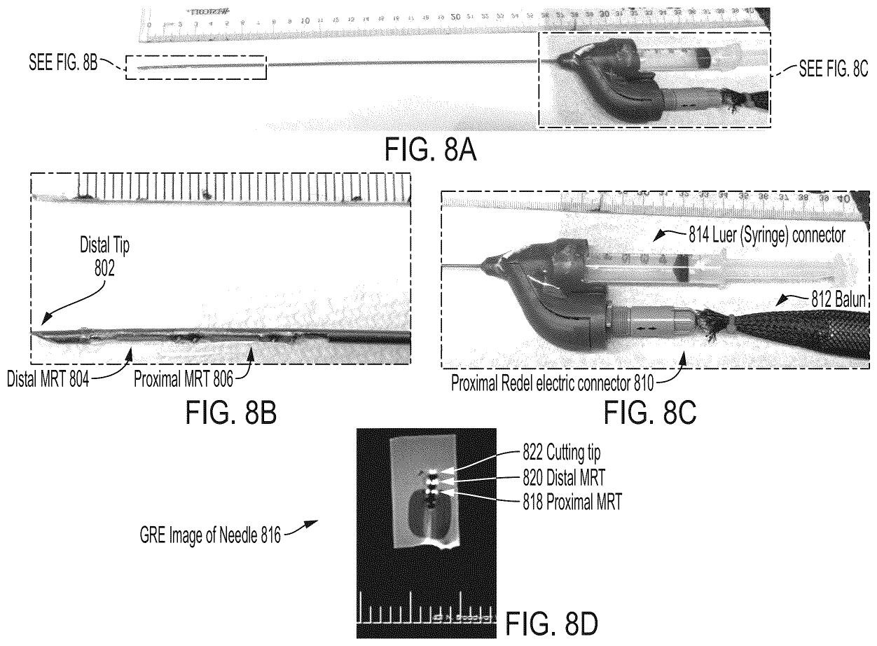

FIG. 8A-8D show a series of pictures of the MR-Tracked injection needle according to examples of the present disclosure, where FIG. 8A shows a complete 24 cm long and 1.6 mm outer diameter needle, FIG. 8B shows an expanded distal end 802 showing the MRT coils 804, 806 and beveled tip, FIG. 8C shows an expanded handle 808, showing syringe 814, quick-disconnect electrical connectors 810, and Balun 812, and FIG. 8D shows a GRE MRI image of needle tip, showing a hyperintense beveled tip 822 and MRT coils 818, 820. The tip hyperintensity and the dark susceptibility artifacts proximal to the MRT coils are caused by the cut titanium surfaces, which are seen only with GRE, and not with TSE images, and can be reduced by coating the metal with an appropriately designed paramagnetic metallic film (e.g. “susceptibility matching”).

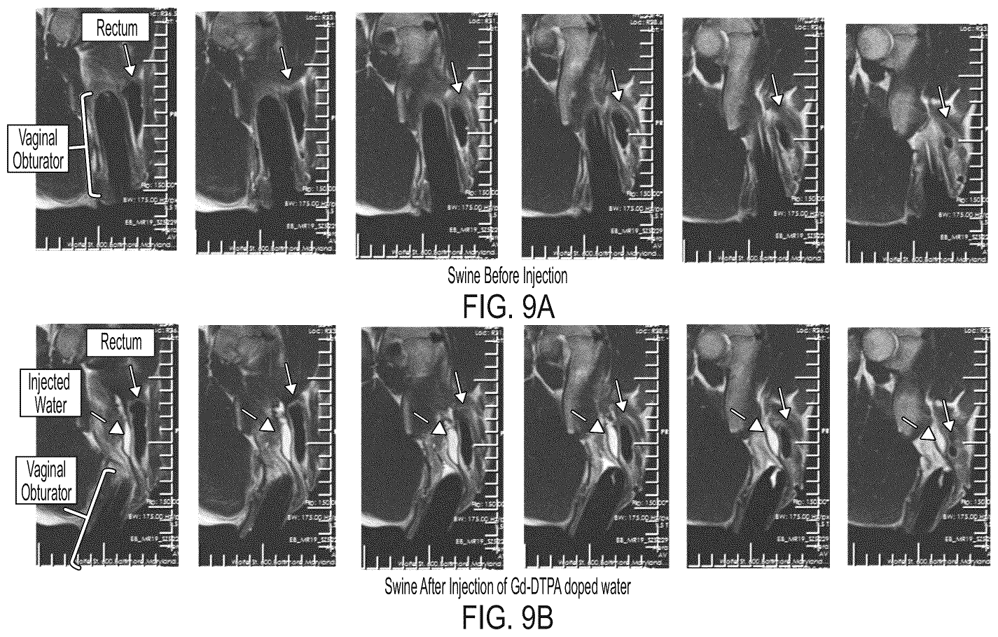

FIGS. 9A and 9B show a series of pictures of MR-guided navigation in a swine, where FIG. 9A shows T2-weighted images acquired before navigation and injection that show the region of the vaginal cavity, with an inserted vaginal obturator, and rectum, and FIG. 9B shows Images obtained after injection of GDTPA-doped water between the vaginal wall and rectum show the topology of the created pocket.

The device(s), as disclosed above, can be used in specific applications in conjunction with (1) MR-tracking pulse sequences; (2) visualization interfaces that enable the visualization of the tracked devices moving within the anatomy, typically with the device shapes overlaid on two-dimensional (2D) or 3D images of the anatomy. The anatomy can be obtained from a variety of MRI contrasts (T1-weighted imaging, T2-weighted imaging, Diffusion Weighted Imaging, MR Angiography, etc.); and (3) Sequential (temporal) acquisitions of 2D or 3D MRI Sequences (Gradient Recalled Echo, T2-weighted imaging) that can be used to quantify the topology and volume of the fluid/hydrogel pocket as it is created.

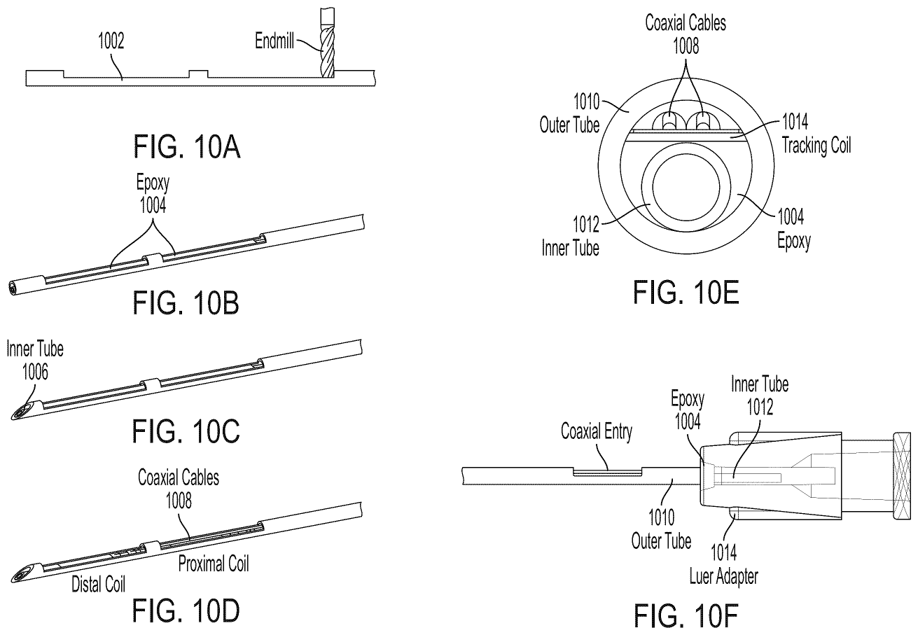

FIG. 10A shows a side view 1002 of the outer tube during the machining operations that are shown in FIG. 10B-FIG. 10D according to examples of the present disclosure. The step-by-step process starts with the application of epoxy (1004 of FIG. 10B), followed by the machining of the beveled tip (1006 of FIG. 10C), and ends with the inclusion of the MRT circuitry, including a distal coil, a proximal coil, and coaxial cables (1008 of FIG. 10D). FIG. 10E shows a cross-section of the MR-tracked needle at the distal end, showing coaxial cables 1008, outer tube 1010, inner tube 1012, tracking coils 1014, and epoxy 1004. FIG. 10F shows a side view of the proximal end of the needle assembly according to examples of the present disclosure.

In some examples, the disclosed injection needle can be fabricated from two 30 cm long titanium tubes (ID: 1.20 mm, OD: 1.60 mm, SKU: TiGr2-TB-040 and ID: 0.61 mm, OD: 0.81 mm, SKU: TiGr2-TB-020). The outer tube's OD can be selected to match the current clinical workflow in MR-guided brachytherapy, mitigating tissue disruption during puncture, and serving as an outer profile for the MRT micro-coils. The outer tube's ID and the inner tube's OD can be sized to provide sufficient space for two micro-coaxial cables (OD: 0.15 mm) to run through the cavity between the outer and inner tube. The inner tube's inner diameter (0.61 mm) can be selected to provide a lumen that was similar in size to a 20-gauge hypodermic needle (0.603 mm), ensuring easy flow and sealed transport of the hydrogel from the syringe to the injection needle tip.

Fabrication of the injection needle assembly can begin with subtractive manufacturing on the outer tube. Two 12.70 mm long grooves can be cut axially along the outer tube using a four-flute, 45°, ⅛-inch carbide end mill (SKU 415-1502, Shars Tool, IL, USA), to provide a placement location for the MRT coils (FIG. 10A). These grooves can be cut to a depth of 0.80 mm to ensure sufficient space was provided within the outer diameter of the outer tube for MRT coil placement. The inner tube can then inserted into the outer tube, and a flat region can be produced for micro-coil placement by filling the gap between the inner and outer tubes at the machined grooves with epoxy (MarineWeld 50172, J B Weld), and then sanding the epoxy flat after curing (FIG. 10B). Following the curing of the epoxy, a 30° bevel tip can be machined onto both nested tubes (FIG. 10C), and the micro-coils were adhered to the epoxy flatbed using fast curing adhesive (FIG. 10D). A detailed view of the front cross-section can be seen in FIG. 10E.

Two micro-coaxial (46-AWG) cables can be allowed entry into the cavity between the inner and outer tube by machining an opening into the outer tube that is 5 mm long and 0.80 mm deep (FIG. 10F). To prevent fluid leakage between the inner and outer tube, the tubes can be attached to a Luer lock (P/N: 11114, Qosina, USA) at the proximal end of the needle using epoxy, with the inner tube extending further into the Luer adapter (FIG. 10F). The entire needle, excluding the distal tip, can be coated by a thin (0.05 mm wall) heat-shrink tube, which prevents contact between the surrounding biological fluids and the circuitry.

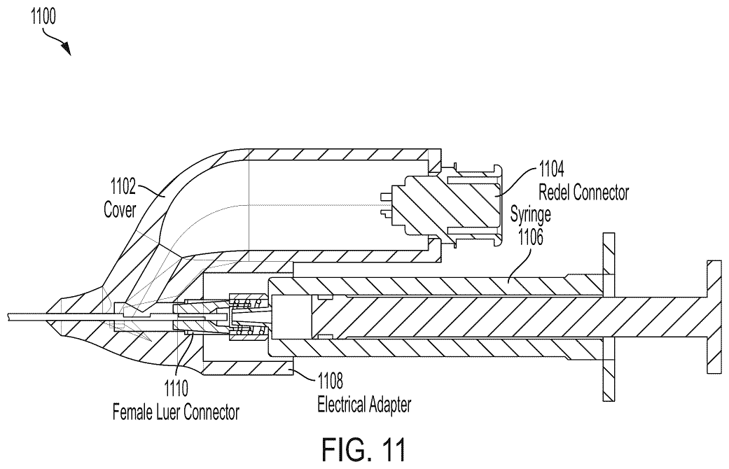

FIG. 11 shows an electrical adapter with the injection needle assembly of FIG. 10E and FIG. 10F. The injection needle assembly can include two connection interfaces: 1) MRT micro-coils to the MRI scanner and 2) injection needle to the hydrogel syringe. The quick connection between the MRT micro-coils and the MRI for tracking signal transmission can be provided through an electrical adapter, which incorporates a quick-disconnect 5-pin Redel (Lemo, Switzerland) connector 1104 that can be covered by cover 1102. To maintain a compact, ergonomic design, the electrical adapter can be made using an SLA printer (Form 3, FormLabs, Massachusetts, USA) that orients the Redel connector to be parallel with the syringe 1106, as shown in FIG. 11. A hollow channel can be created within the electrical adapter to enable easy access to the distal MRT micro-coils. The connection between the injection needle and syringe can be achieved via a Luer connector 1110 connected to an electrical adapter 1108.

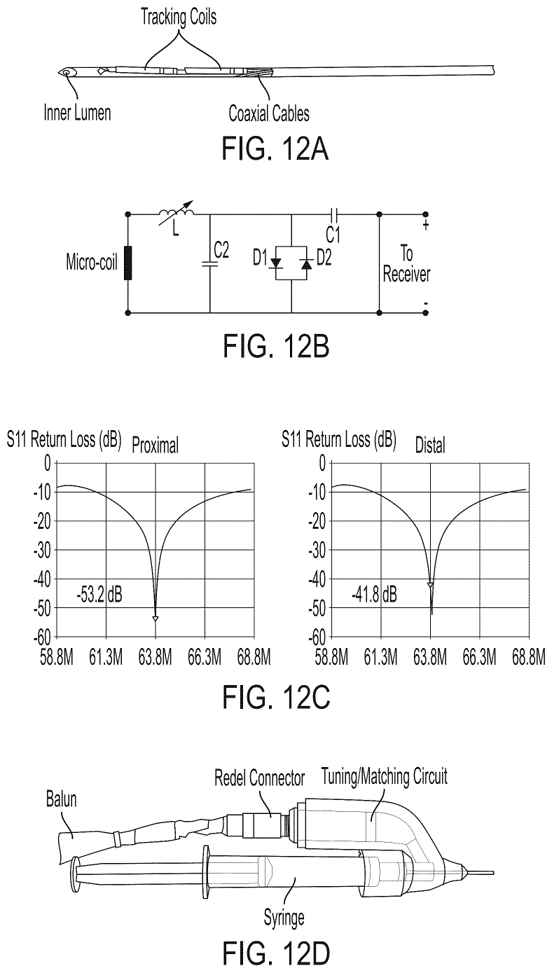

FIG. 12A shows the FPC tracking coils attached to the distal end of the needle according to examples of the present disclosure. At this stage, the coaxial cables have been routed through the space between the inner and outer tubes. FIG. 12B shows the tuning and matching circuit schematic according to examples of the present disclosure. FIG. 12C shows the results of the tuning and matching of the circuit for each coil, indicating strong signal transmission according to examples of the present disclosure. FIG. 12D shows a side view of injection needle and electrical adapter according to examples of the present disclosure.

According to experiments conducted, real-time tracking of the injection needle within the phantom and swine body was achieved by locating two micro-coils (length, width, and thickness: 8 mm, 1.10 mm, and 0.20 mm), made of a 3-layer flexible-printed-circuit (FPC) with embedded capacitors, on the distal end of the needle assembly, as shown in FIG. 12A. The micro-coils are channeled via a tuning, matching, and active decoupling circuit (FIG. 12B) located in the electrical adapter. Tuning and matching are performed using a vector network analyzer (NanoVNA-F, SYSJOINT Information Technology Co., LTD, Zhejiang, China). Series capacitors are used to block DC leakage currents from propagating on the device (as required by International Electrotechnical Commission 60601-1), while parallel capacitors and a variable series inductor (P/N: 164-04A06L, Coilcraft, Illinois, USA) are used to tune the circuit to the 1.5 T MRI Larmor frequency (63.8 MHz). An S11 reflection coefficient of 53.2 and 41.8 dB can be obtained for the proximal and distal MRT coils, respectively (FIG. 12C). The circuit is connected to the Redel connector in the electrical adapter. Prior to the connection to the receiver, the signals propagate on half-wavelength coaxial cables that are overlaid with 30-cm periodic resonant RF traps (Baluns) to attenuate common-mode propagation, reducing cable-heating risk and increasing signal fidelity.

As discussed above, the device(s), as disclosed above, have been experimentally validated by a number of experiments as discussed below. First, the injection needle mechanical performance was evaluated. Finite element analysis (FEA) was performed to analyze the effects of the MRT grooves' geometric makeup on the needle's mechanical performance. This was evaluated by analyzing needle-tip trajectory deviation caused by various groove lengths, including 0 mm (no grooves), 5 mm, 12.7 mm (actual design dimension), and 20 mm. The needle insertion procedure is modeled as a separation process of two tissue bodies connected with a thin cohesive layer. The tissue medium in the simulation was modeled as gelatin, which shares similar mechanical properties of human tissue. The values of the tissue parameters are presented in Table I.

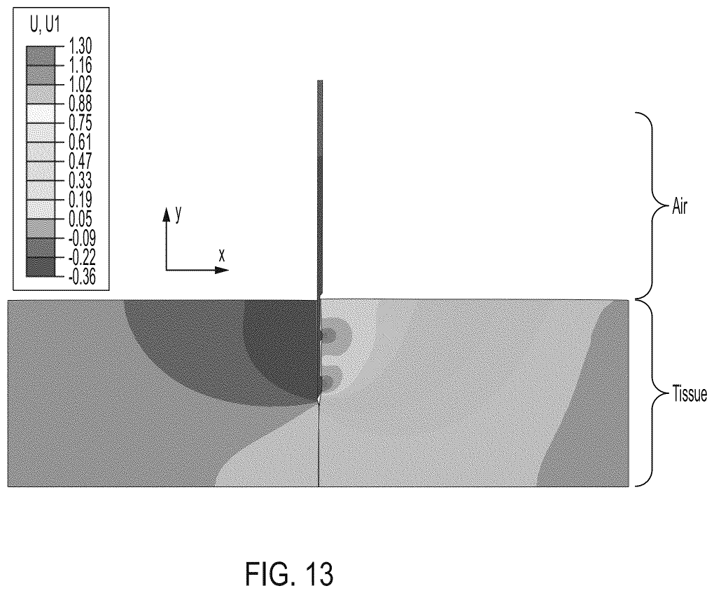

FIG. 13 shows a simulation result of the 30 cm long needle entering a tissue vertically downwards from the top (with 12.7 mm long grooves) when the insertion depth is set to 30 mm according to examples of the present disclosure. The needle successfully eliminates the cohesive element between the two parts of the tissues and separates the tissue into two bodies. Due to the symmetric characteristic of the injection needle, the finite element model was created in 2D to improve computational efficiency (FIG. 13). Herein, the y-direction defines the direction of insertion, and the x-direction quantifies the trajectory deviation during insertion. A static solver was used in the FEA study. For the boundary conditions, the tissue's bottom boundary was vertically fixed and its vertical boundaries were horizontally fixed. The insertion distance of the proximal edge of the needle was −70 mm in the y-direction.

TABLE 1
Simulation Parameters

| Parameters | Units | Values |
|---|---|---|
| Needle Length | mm | 300 |
| Tissue Thickness | mm | 60 |
| Young's Modulus of Tissue | Pa | 7000 |
| Young's Modulus of Needle | GPa | 120 |
| Poisson Ratio of Tissue | — | 0.475 |
| Poisson Ratio of Needle | — | 0.3 |

Second, MRI-guided phantom experiments were performed. Experiments were conducted in a Siemens 1.5 T Espree MRI scanner using a dedicated MR-tracking sequence that reconstructed the needle-tip location and orientation at a rate of ˜15 Hz on the scanner's reconstruction processor. Instantaneous micro-coil positional feedback was sent to a workstation with a 3D Slicer MR-Tracking module [24]. The MR-Tracking module overlaid needle position and orientation on MR navigational roadmaps and displayed the navigational aid on an in-room monitor.

Phantom experiments were performed using a custom prostate gel phantom (gel conductivity=0.6 S/m, gel relative dielectric constant=77). The navigational roadmap was acquired using a T1-weighted Turbo-Spin-Echo (TSE) image dataset array (TR\TE\θ=500 ms\3 ms\90°, ETL=6, 0.6×0.6×3.0 mm³, 64 slice, 2 min acquisition), which combined the scanner abdominal and spine surface arrays along with the MRT coils. MR-Tracking (TR\ TE\θ=2.2 ms\1.1 ms\5°, 0.9×0.9×0.9 mm³ resolution, Hadamard encoding, 5 phase-dithering-directions/projection) was used for needle tracking during the procedure. Once the needle was in position, water was injected into the phantom using a medical syringe and dynamic changes in the injected volume were recorded for 20 seconds using 2D Gradient Recalled Echo (GRE) images (TR\TE\0=10 ms\3 ms\50°, 2.0×2.0×4.5 mm³, 5 slice/sec).

Third, MRI swine experiments were performed. The swine experiment was conducted with institutional IACUC approval in a sexually mature (>6 mth) female Gottingen mini-pig, with a vaginal obturator placed in the vaginal canal, which mimicked the brachytherapy procedure used for gynecologic cancer treatment. The needle was inserted from the open tip of the obturator while observing the navigational workstation. The navigational roadmap was acquired with a T2-weighted TSE dataset (TR\TE\θ=2000 ms\101 ms\180, 0.8×0.8×3.0 mm³). MR-Tracking was used during needle navigation to steer the needle. (0.1 mL/L Gadavist, Bayer Healthcare, USA) Gd-DTPA-doped water was injected into the rectovaginal septum to create the fluid-filled pocket, with images acquired prior to and following pocket creation.

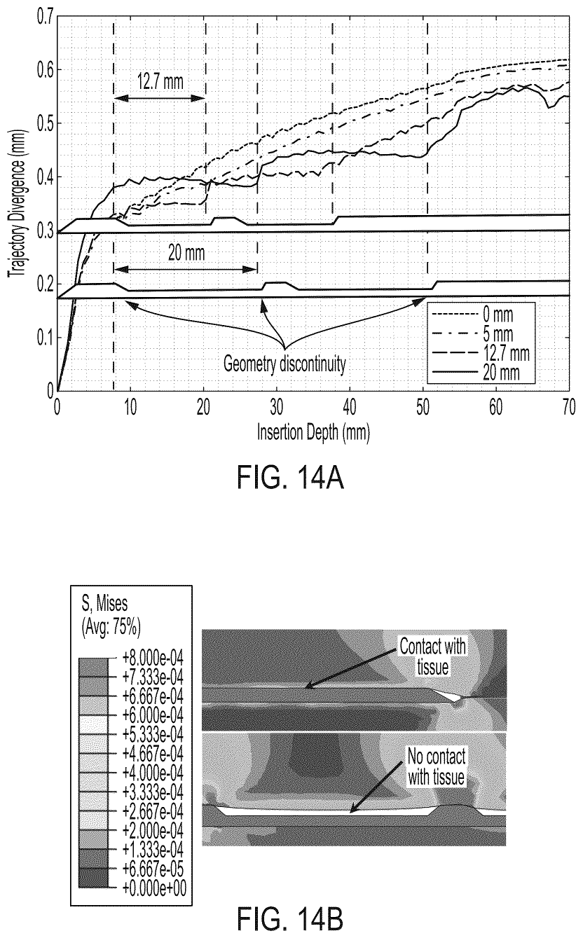

FIG. 14A shows the simulated needle tip deviation from the linear insertion path is plotted with respect to the insertion depth according to examples of the present disclosure. FIG. 14B shows the visual simulation results of a needle shown in FIG. 14A without grooves (FIG. 14A) and FIG. 14C shows the visual simulation results of a needle shown in FIG. 14A with grooves depicted within the tissue with respect to the von Mises stress according to examples of the present disclosure.

Two needle groove geometries are overlaid on the graph, providing a visual aid relating needle geometrical discontinuities to trajectory divergence as they pass the tissue's top boundary (FIG. 13). FIG. 14B shows the visual simulation results of a needle without grooves (FIG. 14B) and with grooves (FIG. 14C) is depicted within the tissue with respect to the von Mises stress. Notice the separation (white) of the tissue surface from the needle with grooves, contributing to the reduction in total trajectory deviation.

The simulation results, as shown in FIG. 14A and FIG. 14B, show that the injection needle successfully separates the two tissue bodies, regardless of groove length. Overall, tip divergence is approximately proportional to the needle insertion depth. This is primarily due to the bevel tip of the injection needle, which tends to re-direct the needle when it is inserted into the tissue, resulting in a tip divergence of the needles' tip at the end of its trajectory of 0.62 mm, 0.61 mm, 0.58 mm, and 0.55 mm for the groove length of 0 mm, 5 mm, 12.7 mm, and 20 mm, respectively. However, the maximum tip divergence is less than 1 mm for the insertion depth of 70 mm (approximately 0.20% of needle length, see FIG. 5A), which can be neglected due to the scanner's typical imaging resolution of 1 mm×1 mm per pixel.

Note that the needle geometry is overlaid onto the trajectory for the needles with the groove length of 12.7 mm and 20 mm in FIG. 5A to provide a visual aid. Although a larger groove length tends to reduce the overall strength while the cut shaft remains partially outside the tissue, causing a larger deviation during the first 8 mm of insertion, it also reduces the contact area between the needle and surrounding tissue after the groove geometry is within the tissue (FIG. 5B), leading to a smaller resultant force in the negative x-direction (coordinate system convention in FIG. 4). Therefore, the simulation studies also indicate that the maximum tip divergence is not directly proportional to the groove length.

From the MRI phantom experiment, MR-tracking precision was determined by comparing the ground-truth location of the MRT coil positions to the 3D-Slicer MR Tracking module in the prostate phantom (CIRS, VA, USA). Ground-truth positions were measured using high-resolution 3D MR images acquired by an inversion-recovery gradient echo (MP-RAGE) sequence with a spatial resolution of 0.3×0.3×0.3 mm³. MRT SNR supported 15 Hz navigation with tracking precision of 0.9×0.9×0.9 mm³ and robust visualization of the tip location and orientation.

To validate dynamic pocket topology visibility, 30 mL of Gd-DTPA-doped water was injected between the prostate and rectum (see FIG. 6 for the created pocket in the prostate phantom). The dynamic changes in the volume of the injected water were clearly captured, providing real-time feedback of the injected fluid topology. During the injection procedure, the syringe was swapped out 5 times for injection of additional fluid; however, the needle tip only moved 2 mm, indicating intra-procedural syringe replacement is easy and safe.

In the MRI swine experiment, the injection needle was evaluated in a live swine. MR-Tracked navigation was successfully used to place the injection needle between the vagina and rectum of the swine (FIG. 7). The needle was repositioned 2 times within 20 seconds to achieve the desired targeting performance, but the trajectories were linear, indicating our model was accurate in predicting negligible tip divergence. Note that the existing approaches to create the pocket rely on passive tracking, which has a significantly longer procedure time due to slow image feedback during clinician deployment. The animal study performed with the proposed injection needle encourages prompt clinical implementation due to its fast-tracking and ease of use, resulting in significantly shorter procedure time.

After the injection needle placement, T2-w imaging was performed to monitor the vaginal wall, rectum, and pocket. The dynamic visualization of injected Gd-DTPA-doped water pocket topology is shown in FIG. 7. A total of 15 mL of water was injected, resulting in an additional spacing of 12±2 mm between the vagina and rectum.

In summary, the design and fabrication, simulation modeling, and MRI phantom validation of a custom-designed MR-Tracked injection needle for hydrogel pocket placement is disclosed. MRT micro-coil integration was successfully implemented and provided real-time tracking of the distal end of the needle with an accuracy of 0.9×0.9×0.9 mm³ at 15 Hz. The simulation indicated that the bevel tip and MRT coil grooves machined onto the needle had a minimal effect on trajectory deviation, with a limited 0.52 mm deviation in the direction perpendicular to insertion for a tissue insertion depth of 60 mm. Furthermore, the metallic-shaft grooves, created for MRT coil placement, reduced the trajectory deviation. The actively-tracked injection needle was successful at displacing the vagina from the rectum in the swine model by 12±2 mm in the anterior-posterior direction within 20 seconds.

Notwithstanding that the numerical ranges and parameters setting forth the broad scope of the present teachings are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 5. In certain cases, the numerical values as stated for the parameter can take on negative values. In this case, the example value of range stated as “less than 10” can assume negative values, e.g. −1, −2, −3, −10, −20, −30, etc.

While the present teachings have been illustrated with respect to one or more implementations, alterations and/or modifications can be made to the illustrated examples without departing from the spirit and scope of the appended claims. For example, it will be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or implementations of the present teachings. It will be appreciated that structural components and/or processing stages can be added or existing structural components and/or processing stages can be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items can be selected. As used herein, the term “one or more of” with respect to a listing of items such as, for example, A and B, means A alone, B alone, or A and B. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “about” indicates that the value listed may be somewhat altered, as long as the alteration does not result in nonconformance of the process or structure to the illustrated implementation. Finally, “exemplary” indicates the description is used as an example, rather than implying that it is an ideal. Other implementations of the present teachings will be apparent to those skilled in the art from consideration of the specification and practice of the disclosure herein. It is intended that the specification and examples be considered as exemplary only, with a true scope and spirit of the present teachings being indicated by the following claims.

What is claimed is:

1. A magnetic resonance Imaging (MRI)-tracked injection needle device comprising:
a luer syringe;
an electrical connector that is at least partially housed in an interior space of a distal end of the luer syringe;
an electrical adaptor coupled to the electrical connector; and
an injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip and comprising one or more tracking coils arranged between the inner metal tube and the outer metal tube.

2. The magnetic resonance Imaging (MRI)-tracked injection needle device of claim 1, wherein the needle distal end further comprising two holes cut into the outer metal tube to permit MRI signals to be obtained from more than one orientation of the injection needle.

3. The magnetic resonance Imaging (MRI)-tracked injection needle device of claim 1, wherein the tip is a beveled tip.

4. The magnetic resonance Imaging (MRI)-tracked injection needle device of claim 3, wherein the beveled tip has a 30° bevel.

5. The magnetic resonance Imaging (MRI)-tracked injection needle device of claim 1, wherein the needle distal end further comprising flexible printed circuit board connected by cables to the one or more tracking coils.

6. The magnetic resonance Imaging (MRI)-tracked injection needle device of claim 5, wherein the one or more tracking coils provide electrical tracking signals representative of location information of the tip and the flexible printed circuit board transmits the electrical tracking to a receiver of a MRI scanner.

7. The magnetic resonance Imaging (MRI)-tracked injection needle device of claim 1, wherein the needle distal end further comprising a protective heat shrink to provide water insulation for the one or more tracking coils and cables.

8. The magnetic resonance Imaging (MRI)-tracked injection needle device of claim 1, wherein the concentric metal tubes are composed of titanium.

9. The magnetic resonance Imaging (MRI)-tracked injection needle device of claim 1, wherein the inner metal tube has dimensions of 0.81 mm×0.10 mm and the outer metal tube has dimensions of 1.62 mm×0.2 mm.

10. The magnetic resonance Imaging (MRI)-tracked injection needle device of claim 1, wherein the outer metallic tube contains groves of defined dimensions and at specific locations in order to increase or decrease the trajectory linearity or deviation when the injection needle is inserted into a specific tissue.

11. The magnetic resonance Imaging (MRI)-tracked injection needle device of claim 1, further comprising an insulator layer arranged on an exterior surface of the outer metal tube.

12. The magnetic resonance Imaging (MRI)-tracked injection needle device of claim 10, wherein the shaft further comprises a loosely wound solenoid with a pitch greater than one that is connected in series to a thin film capacitor that is separated by the insulator layer with one end of the solenoid and one end of the thin film capacitor to the outer metal tube to reduce MRI radio-frequency induced heating of the shaft.

13. A method for fabricating a magnetic resonance Imaging (MRI)-tracked injection needle device, the method comprising:
attaching an injection needle to a syringe, the injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip;
disposing at least one coil of electrical conductor between the inner metal tube and the outer metal tube; and
providing an electrical output to at least one coil.

14. A method for using a tracking system for magnetic resonance imaging (MRI), the method comprising:
electrically connecting an injection needle to electronic circuitry, the injection needle comprising a shaft having a needle distal end and a needle proximal end, the shaft comprising concentric metal tubes comprising an inner metal tube and an outer metal tube, the needle proximal end coupled to the electrical adaptor and the needle distal end terminating at a tip and comprising a first tracking coil and a second tracking coil arranged between the inner metal tube and the outer metal tube;
generating sequences of MRI-pulses to acquire, with an MRI system, projection data representing three one-dimensional projections of the injection needle along three orthogonal spatial axes such as to determine a three-dimensional position of the at least one coil of the first tracking coil and the second tracking coil from sequenced projection data; and
generating data representing a position of the tip by extrapolating the projection data along a direction connecting positions of the first tracking coil and the second tracking coil.
